(19) United States
(12) Patent Application Publication
(10) Pub. No.: US 2025/0060310 A1
(43) Pub. Date: Feb. 20, 2025

(54) METHOD FOR ANALYZING ALIASED SPECTRA OF ALKANE GASES

(71) Applicant: Hefei Institutes of Physical Science, CAS, Hefei (CN)

(72) Inventors: Zhirong Zhang, Hefei (CN); Yuntao Peng, Hefei (CN); Hua Xia, Hefei (CN); Pengshuai Sun, Hefei (CN); Qianjin Wang, Hefei (CN); Tao Pang, Hefei (CN); Bian Wu, Hefei (CN)

(73) Assignee: Hefei Institutes of Physical Science, CAS, Hefei (CN)

(21) Appl. No.: 18/802,366

(22) Filed: Aug. 13, 2024

(30) Foreign Application Priority Data
Aug. 16, 2023 (CN) 202311028017.3

(51) Int. Cl. G01N 21/39; G06F 17/18

(52) U.S. Cl. CPC G01N 21/39; G06F 17/18

(57) ABSTRACT

Provided is a method for analyzing aliased spectra of alkane gases, including the following steps: firstly separately acquiring second harmonic signals of specified gases at known gas concentrations and calculating an absorbance of each gas; subsequently constructing a low concentration prediction model regarding second harmonic signals and gas concentrations; next constructing a high concentration prediction model regarding absorbances and gas concentrations, immediately followed by acquiring a second harmonic signal of a gas to be measured with an unknown gas concentration and calculating an absorbance of the gas to be measured; and if an absorbance peak of the gas to be measured is less than or equal to a threshold A₀, predicting the gas concentration of the gas to be measured using the low concentration prediction model; otherwise, predicting the gas concentration of the gas to be measured using the high concentration prediction model.

### CROSS REFERENCE TO RELATED APPLICATION

This patent application claims the benefit and priority of Chinese Patent Application No. 202311028017.3, filed with the China National Intellectual Property Administration on Aug. 16, 2023, the disclosure of which is incorporated by reference herein in its entirety as part of the present application.

### TECHNICAL FIELD

The present disclosure relates to the technical field of gas detection, and in particular, to a method for analyzing aliased spectra of alkane gases.

### BACKGROUND

Some important components, such as C₃H₈ and C₄H₁₀, in liquefied petroleum gas are colorless, flammable, and combustible. C₃H₈ and C₄H₁₀ are gathered in a hollow due to large densities thereof so as to form a high concentration gathering area prone to explosion. Therefore, it is highly necessary to detect the concentrations of C₃H₈ and C₄H₁₀ in a petroleum storage site.

At the present stage, the concentrations of gases are mainly detected by chemical sensors in a petroleum industry site. However, traditional chemical sensors are limited to particular detection elements and are susceptible to a temperature and a humidity of an external environment. Such traditional chemical sensors may usually have low detection accuracy and need to be frequently replaced. To avoid this problem, patent No. 201610896383.4 discloses a dual-optical-path modulation detection method in infrared spectroscopy gas logging that utilizes characteristic absorption of gas molecules within a range of a certain wave band to analyze and identify categories of gases and predicts a concentration of a gas to be measured based on an absorption spectral intensity. In combination with a long-optical path gas absorption cell technique, system stability can be well improved by using an alternate flow type gas circuit modulation technique. However, organic compounds such as C₃H₈ and C₄H₁₀ have complex molecule structures, and there is a high probability that infrared absorption spectra formed by rotation of molecular functional functions and vibration of chemical bonds will be superposed with one another, resulting in a widened characteristic absorption spectrum of molecules. The occurrence of continuous wide-spectrum absorption characteristics within a certain range may have a direct impact on a measurement result, resulting in reduced accuracy of a detection result.

### SUMMARY

To avoid and overcome the technical problems in the prior art, the present disclosure provides a method for analyzing aliased spectra of alkane gases. The present disclosure can allow for demodulation of aliased spectra of C₃H₈ and C₄H₁₀ so as to accurately obtain the gas concentration information of each component of the mixed gas.

In order to achieve the above objective, the present disclosure provides the following technical solution:

A method for analyzing aliased spectra of alkane gases includes the following steps:
S1, separately acquiring second harmonic signals of specified gases at known gas concentrations and calculating an absorbance of each gas;
S2, constructing a low concentration prediction model regarding second harmonic signals and gas concentrations;
S3, constructing a high concentration prediction model regarding absorbances and gas concentrations;
S4, acquiring a second harmonic signal of a gas to be measured with an unknown gas concentration and calculating an absorbance of the gas to be measured; and
S5, if an absorbance peak of the gas to be measured is less than or equal to a threshold A₀, predicting the gas concentration of the gas to be measured using the low concentration prediction model; otherwise, predicting the gas concentration of the gas to be measured using the high concentration prediction model.

As a further solution of the present disclosure, the specified gases may include a single gas C₃H₈, a single gas C₄H₁₀ and a mixed gas of the single gas C₃H₈ and the single gas C₄H₁₀ in a set ratio; and the threshold A₀ may be 0.4.

As a further solution of the present disclosure, step S1 includes the following specific steps:
S11, establishing an absorption cell having an optical path of L and emitting a triangular scanning signal from an incident end of the absorption cell into the absorption cell;
S12, subsequently bubbling N₂ into the absorption cell to create a N₂ atmosphere, and acquiring a N₂ second harmonic signal produced after irradiating N₂ with triangular scanning signals of different wavelengths, and taking the N₂ second harmonic signal as a light intensity baseline;
S13, subsequently bubbling specified gases with different known gas concentrations into the absorption cell separately;
S14, using triangular scanning signals of same wavelengths as those in step S12 to individually irradiate the specified gases bubbled into the absorption cell, respectively, and obtaining a second harmonic signal formed after the irradiation; and
S15, finally calculating absorbances of each single gas and the mixed gas.

As a further solution of the present disclosure, the low concentration prediction model and the high concentration prediction model are each established by partial least squares regression.

As a further solution of the present disclosure, when the low concentration prediction model is established using partial least squares, a second harmonic signal is used as an independent variable and a low concentration independent variable matrix X₁^{n×m} is established, where n represents a number of samples; m represents a number of feature points, namely types of triangular scanning signal wavelengths; and with a gas concentration as a dependent variable, a dependent variable matrix Y₁^{n×p} is established, where p represents a category of a gas concentration.

As a further solution of the present disclosure, when the high concentration prediction model is established using partial least squares, an absorbance is used as an independent variable and a high concentration independent variable matrix X₂^{n×m} is established, where n represents a number of samples; m represents a number of feature points, namely types of triangular scanning signal wavelengths; and a gas concentration is used as a dependent variable and a dependent variable matrix Y₂^{n×p} is established, where p represents a category of a gas concentration.

As a further solution of the present disclosure, a calculation formula for the absorbance of a gas with a known gas concentration is as follows:

$${A}_{1} - ln ( \frac{1}{exp [ - S ( T ) CPL φ ( v ) ]} )$$

where A₁ represents the absorbance of the gas with the known gas concentration; v represents a wave number of an absorption line spectrum; S(T) represents a line intensity corresponding to a gas temperature of T; C represents the gas concentration; p represents a total pressure of the gas; φ(v) represents a linear absorption function of integral area normalization; and L represents the optical path of the absorption cell.

As a further solution of the present disclosure:
a calculation formula for the absorbance of a gas with an unknown gas concentration is as follows:

$${A}_{2} = ln ( \frac{{I}_{0}}{{I}_{t}} )$$

where A₂ represents the absorbance of the gas with the unknown gas concentration; I₀ represents an initial light intensity of incident light; and Iₜ represents an outgoing light intensity of the incident light.

As a further solution of the present disclosure, a second harmonic signal is produced by the following process: a triangular scanning signal is emitted from the incident end of the absorption cell into the absorption cell to be absorbed by a gas, and then received and converted by a photoelectric detector integrated at an exit end of the absorption cell into an electric signal, and finally, the electric signal is demodulated by a lock-in amplifier to output the corresponding second harmonic signal.

As a further solution of the present disclosure, the absorbance peak of the gas to be measured is obtained by the following process: an infrared laser, used as a triangular scanning signal, is emitted from the incident end of the absorption cell into the absorption cell to be absorbed by a gas, and then received and converted by the photoelectric detector integrated at the exit end of the absorption cell into an infrared spectrum, and finally, the corresponding absorbance peak is obtained from the infrared spectrum.

Compared with the prior art, beneficial effects of the present disclosure are as follows:

1. The present disclosure can allow for demodulation of aliased spectra of the single gas C₃H₈ and the single gas C₄H₁₀ so as to accurately obtain the gas concentration information of each component of the mixed gas. The present method covers a wide measuring span. After compartmental modeling, the single gas C₃H₈ in a concentration range of 0.8%-100% LEL and the single gas C₄H₁₀ in a concentration range of 0.9%-100% LEL can be accurately measured. The requirement of online monitoring on a plurality of gases in a petroleum industry site can be met.

2. The present disclosure accomplishes simultaneous online monitoring of the single gas C₃H₈ and the single gas C₄H₁₀ of petroleum volatile gases. The demodulation of the aliased absorption spectra of the single gas C₃H₈ and the single gas C₄H₁₀ can be completed by partial least squares regression. By piecewise switching the concentration prediction models, the concentration prediction model established based on a second harmonic signal and a triangular scanning signal is used at a low concentration with an absorbance of less than 0.4. At a high concentration with an absorbance of greater than 0.4, the concentration prediction model established based on ab absorbance signal is used. Thus, the influences of a light intensity baseline drift and nonlinearity of the second harmonic signal are reduced.

### BRIEF DESCRIPTION OF THE DRAWINGS

FIG. 1 is a flowchart of main operation steps according to the present disclosure;

FIG. 2 is a structural schematic diagram of an experimental system according to the present disclosure;

FIG. 3 is a flowchart of detailed operation steps according to the present disclosure;

FIG. 4A is a prediction result chart for a single gas C₃H₈ according to the present disclosure;

FIG. 4B is a prediction error chart for the single gas C₃H₈ according to the present disclosure;

FIG. 4C is a prediction result chart for a single gas C₄H₁₀ according to the present disclosure;

FIG. 4D is a prediction error chart for the single gas C₄H₁₀ according to the present disclosure;

FIG. 5A is a prediction result chart for C₃H₈ in a mixed gas according to the present disclosure;

FIG. 5B is a prediction error chart for C₃H₈ in the mixed gas according to the present disclosure;

FIG. 5C is a prediction result chart for C₄H₁₀ in the mixed gas according to the present disclosure; and

FIG. 5D is a prediction error chart for C₄H₁₀ in the mixed gas according to the present disclosure.

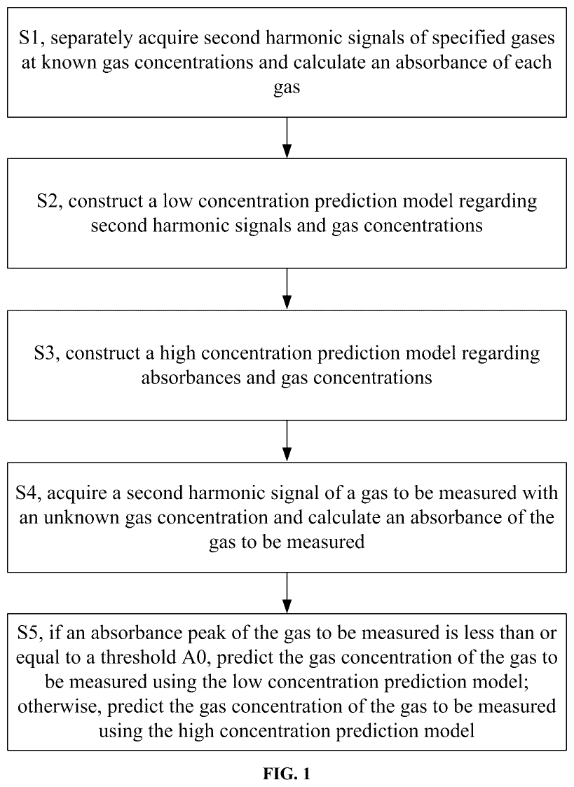

List of Reference Numerals: 1—signal generator; 2—driver board; 3—distributed feedback laser device; 4—absorption cell; 5—photoelectric detector; 6—lock-in amplifier; and 7—display.

### DETAILED DESCRIPTION OF THE EMBODIMENTS

The technical solutions of the embodiments of the present disclosure are clearly and completely described below with reference to the drawings in the embodiments of the present disclosure. Apparently, the described embodiments are merely a part rather than all of the embodiments of the present disclosure. All other embodiments derived from the embodiments in the present disclosure by a person of ordinary skill in the art without creative efforts shall fall within the protection scope of the present disclosure.

### 1. EXPERIMENTAL SYSTEM

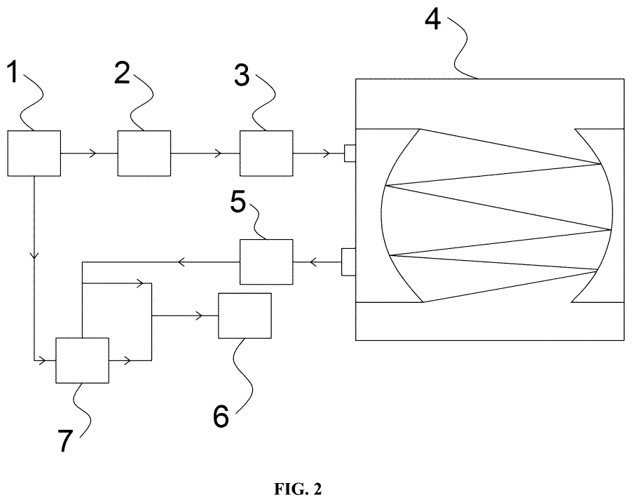

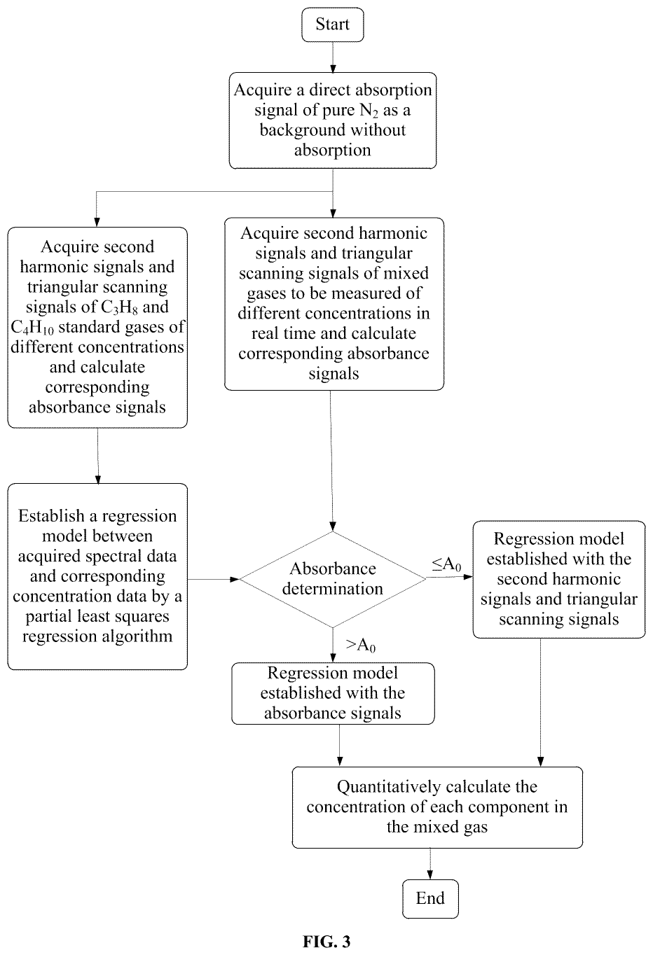

The experimental system is as shown in FIG. 2. In the experiment, a distributed feedback laser device 3 with a central wavelength of 1,686.0 nm is used, which is controlled by a corresponding laser device driver board 2, and the driver board 2 is controlled by a signal generator 1. The signal generator 1 generates a low-frequency triangular scanning signal at a frequency of 10 Hz and a high-frequency sinusoidal modulating signal at a frequency of 10 KHz. In each scanning period, a driving current signal in a first half period only contains the low-frequency triangular scanning signal, and in the second half period, a high-frequency sinusoidal signal is utilized to modulate the low-frequency triangular scanning signal to a high frequency. An output wavelength of the laser device is tuned and modulated by periodically changing the driving current to cover central positions of absorption peaks of C₃H₈ and C₄H₁₀. The modulated laser is collimated by an optical fiber collimator and then enters into the absorption cell 4. The absorption cell 4 has a basic length of 24.6 cm and a mirror surface diameter of 6 cm, and is covered with a high-reflective dielectric film. The absorption tool 4 has a volume of about 0.8 L and an optical path L of 52.8 m. The laser is absorbed by a sample gas and then received and converted by a photoelectric detector 5 integrated at an exit end of the absorption cell 4 into an electric signal, and the electric signal is demodulated by a lock-in amplifier 6 to output the corresponding second harmonic signal which is presented by a display 7. Subsequently, the acquired spectral data is subjected to modeling analysis and concentration prediction.

### 2. PARTIAL LEAST SQUARES (PLS) ALGORITHM

PLS is a novel multivariate data analysis method that is compatible with the characteristics of principal component regression and linear regression, can effectively process multiple correlations of data, and can fully extract features of an independent variable and a dependent variable with a finite number of samples to obtain a regression coefficient matrix.

When a concentration prediction model is established, it is assumed that there is a total of n groups of samples; X represents a spectral data matrix n×p including p feature points, and p feature points refer to p second harmonic signals produced corresponding to p different wavelengths. Y represents a gas concentration matrix n×q including q dependent variables. By partial least squares regression, a principal component t₁ is extracted in X and a principal component u₁ is extracted in Y. Regressions of X to t₁ and Y to u₁ are then ascertained separately. Subsequently, a second round of component extraction is carried out with residual information after X being explained by t₁ and residual information after Y being explained by u₁. It is repeated in this way until acceptable accuracy is reached. If m principal components t₁, t₂, . . . , tₘ are ultimately extracted for X, by partial least squares regression, regressions of yₖ to t₁, t₂, . . . , tₘ are expressed as regression equations of yₖ with respect to original variables X₁, X₂, . . . , Xₘ, where k=1, 2, . . . , q.

The extracted m principal components form matrices W and U, respectively, and X and Y may be written as:

X=WP^{T}

Y=UQ^{T}

P is an orthogonal matrix of X, and Q is an orthogonal matrix of Y; dimensions of the matrices W and Q are q×m; and dimension of the matrices P and U are p×m. W, U, P, and Q may be all calculated by a nonlinear iterative partial least squares algorithm, and a regression coefficient matrix is calculated by linear regression:

B_{PLS}=(W^{T}W)⁻¹W^{T}U

A spectral data matrix X_{new} of a gas to be measured with any gas concentration is acquired, and the gas concentration Y_{new} thereof is calculated with the known regression coefficient matrix B_{PLS}:

Y_{new}=X_{new}PB_{PLS}Q^{T}

### 3. GAS CONCENTRATION PREDICTION

### 3.1. Gas Concentration Prediction of Single Gas

To preliminarily verify the feasibility of the PLS algorithm for predicting the concentrations of the C₃H₈ and C₄H₁₀ gases, prediction at a low concentration is carried out for pure C₃H₈ gas and pure C₄H₁₀ gas separately.

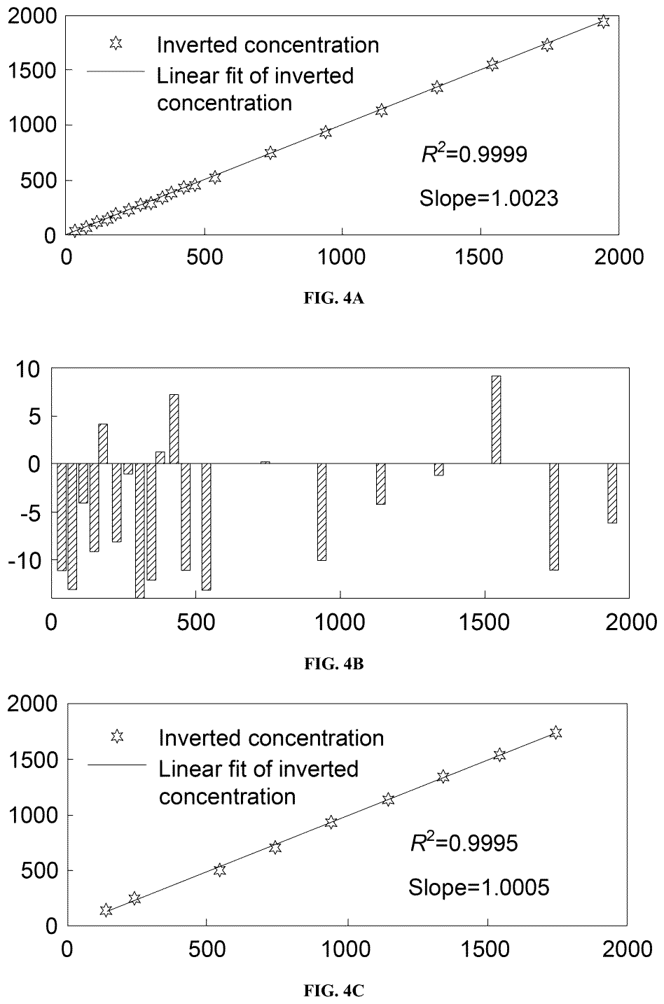

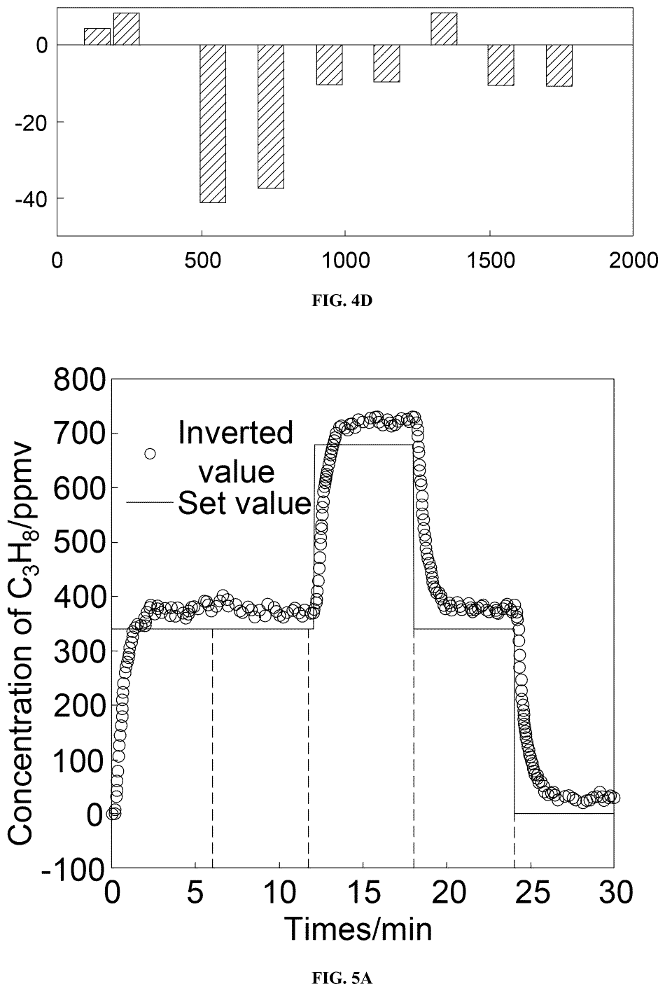

The PLS algorithm is utilized to perform regression modeling on the second harmonic signal data and the gas concentration data of C₃H₈ and C₄H₁₀. A training set is formed by standard gas concentrations within a range of 100 ppmv to 2,000 ppmv, with a step change concentration of 100 ppmv. That is, by the concentrations of 100 ppmv, 200 ppmv, 300 ppmv, . . . , 2,000 ppmv, there are a total of 20 sets of standard gas concentration data. Each set of data records a total of 900 feature points of the second harmonic signals thereof. That is, the independent variable matrix X is 20×900 and the dependent variable matrix Y is 20×1, which are concentrations corresponding to the groups of spectra. During modeling, an optimal number of principal components extracted is selected according to cross effectiveness, and finally, the determined gas concentration prediction model is utilized to perform regression calculation separately on pure C₃H₈ and pure C₄H₁₀ with known concentrations. The calculation results for pure C₃H₈ are as shown in FIG. 4A. In FIG. 4A, Inverted concentration represents the gas concentration calculated by the gas concentration prediction model, and Linear fit of inverted concentration represents a fitted curve of the gas concentration calculated by the gas concentration prediction model. Prediction errors corresponding to pure C₃H₈ are as shown in FIG. 4B. The calculation results for pure C₄H₁₀ are as shown in FIG. 4A, and prediction errors corresponding to pure C₄H₁₀ are as shown in FIG. 4D. As can be seen in FIG. 4A to FIG. 4D, at a low concentration of below 2,000 ppmv, the maximum prediction errors for C₃H₈ and C₄H₁₀ are 14 ppmv and 41 ppmv, respectively, and the relevant coefficient R² is 0.9999 and 0.9995. Thus, in the pure C₃H₈ gas and the pure C₄H₁₀ gas, the prediction model established by partial least squares regression has a good concentration prediction effect for both gases. Since a relative absorption peak of C₄H₁₀ is much smaller than that of C₃H₈, the second harmonic signal demodulated at a low concentration may be greatly affect by a background change due to a small amplitude thereof. Therefore, the prediction errors are relatively large, but still in an acceptable range.

### 3.2. Gas Concentration Prediction of Mixed Gas

The volatile components of petroleum products produced in various places are complex and different from one another, but mainly contain C₃H₈ and C₄H₁₀. Therefore, it is necessary to detect the concentration of each component gas of a mixed gas of both. After the prediction capability of the ns algorithm for the concentrations of the pure C₃H₈ gas and the pure C₄H₁₀ gas is preliminarily verified, the working performance of the established concentration prediction model under the mutual influence of the mixed gas of both needs to be considered. When a single gas is measured, the output wavelength of the laser device may be tuned separately to cover the absorption centers of C₃H₈ and C₄H₁₀. In the mixed gas, limited by the tuning range of the laser device and with overall consideration of influencing factors, a modulation fashion of entirely scanning the absorption peaks of C₄H₁₀ and partially scanning the absorption peaks of C₃H₈. The amplitudes of the second harmonic signals demodulated from C₃H₈ and C₄H₁₀ of a same concentration differ by two orders of magnitude, which does not affect the prediction of the respective gas concentration under the condition of a pure gas. However, in the case of the mixed gas, a C₄H₁₀ signal of a small amplitude will be drown in a huge change of a C₃H₈ signal, and a non-negligible pull-down effect is produced on the amplitude of the second harmonic signal of the absorption center of C₃H₈. Therefore, the separate modeling of second harmonic signal will produce an unacceptable error. Accordingly, it is considered that characteristic absorption information represented by the second harmonic signal is combined with spectral band absorption information represented by a direct absorption signal to act as a feature point for training the concentration prediction model for the mixed gas.

It needs to be noted that in traditional gas measurement of a single absorption peak, the original light intensity of the absorption part is often obtained by a baseline fitting method. In spectral band absorption of a macromolecular alkane gas, the light intensity baseline obtained by fitting is often wrong due to the absorption effect of the whole scanning range. For this, N₂ needs to be bubbled for 5 minutes before each experiment, and an average triangular scanning signal after 32 experiments is taken as the basic light intensity, i.e., the light intensity baseline. Thus, the direct absorption signal of each group of mixed gas is obtained. To improve the prediction accuracy of the model, two groups of parallel experiments of low concentrations 100-800 ppmv and high concentrations 2,000-10,000 ppmv are set.

Prediction groups of the two groups of experiments are set in combination with the contents of both gases in actual oil gas volatiles, and cases in which C₃H₈ and C₄H₁₀ are mixed in different ratios are taken into consideration. The model obtained by mixed gas training has good prediction accuracy for both gases under the conditions of mixing C₃H₈ and C₄H₁₀ in ratios of 1:0, 0:1, 1:1, 2:1, and 1:2. In low concentration groups, the relevant coefficient R² is 0.9985 and 0.9969 for C₃H₈ and C₄H₁₀, respectively, and the maximum absolute errors of prediction are 34 ppmv and 51 ppmv, respectively. In high concentration groups, the relevant coefficient R² is 0.9999 for both C₃H₈ and C₄H₁₀, and the maximum absolute errors are 64 ppmv and 148 ppmv. The prescribed lower explosive limit (LEL) in the industry is 2.1% and 1.9% for C₃H₈ and C₄H₁₀, respectively. The above concentration prediction model not only can detect a low-concentration mixed gas of C₃H₈ 0.8% LEL (168 ppmv) and C₄H₁₀ 0.9% LEL (171 ppmv), but also has the prediction errors of less than 3% LEL at high concentrations, which meets the requirement of production safety.

### 3.3. Analysis of Prediction Performance

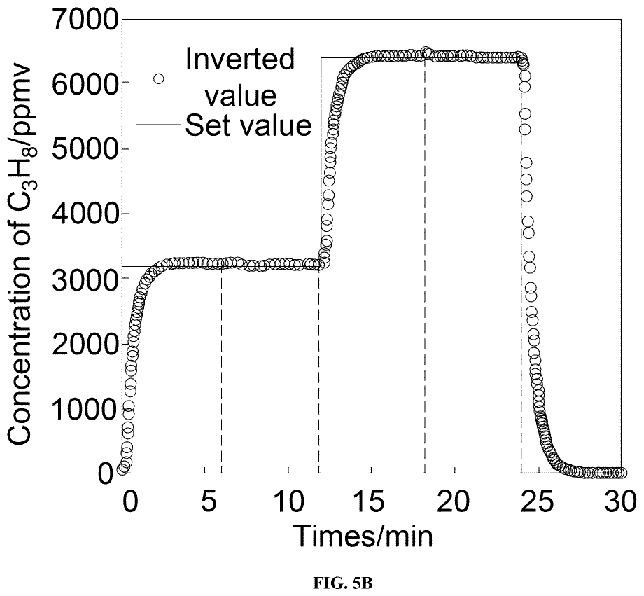

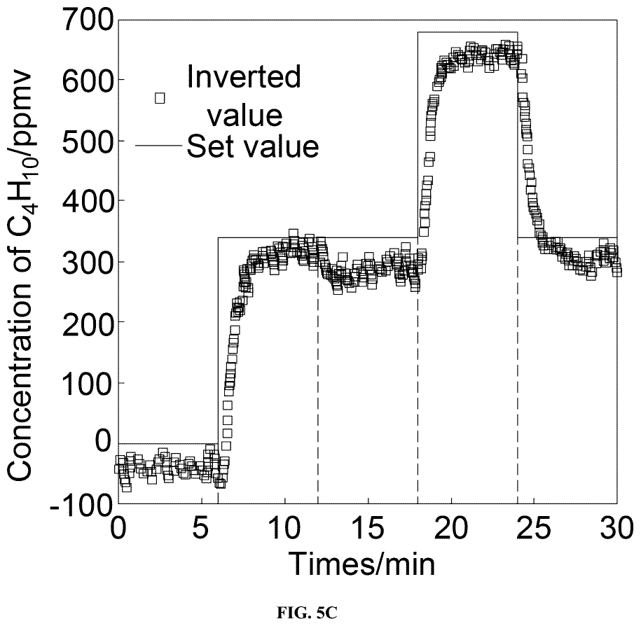

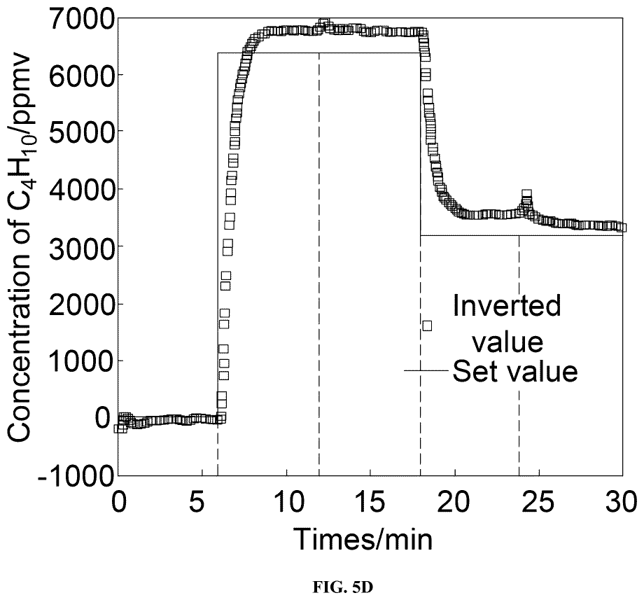

In order to further verify the dynamic reliability of the established mixed gas concentration prediction model when working continuously, two continuous concentrations tests at high and low concentrations are conducted as well. For the low concentration groups, five groups of sample gases of C₃H₈ (ppmv)-C₄H₁₀ (ppmv) at concentrations of 340-0, 340-340, 680-340, 340-680, and 0-340 are configured by a dynamic gas distributor. For the high concentration groups, five groups of sample gases of C₃H₈ (ppmv)-C₄H₁₀ (ppmv) at concentrations of 3,200-0, 3,200-6,400, 6,400-6,400, 6,400-3,200, and 0-3,200 are configured. Each group of sample gases is blown for 6 minutes to ensure that the whole absorption cell 4 is thoroughly and uniformly full of the gases. Real-time prediction of two mixed gases in the whole process is realized according to the models described above. The prediction results of C₃H₈ in the low concentration groups are as shown in FIG. 5A, and the prediction results of C₃H₈ in the high concentration groups are as shown in FIG. 5B. Inverted value represents a gas concentration value predicted by using the gas prediction model, and Set value represents a set value of an actual gas concentration of a gas. The prediction results of C₄H₁₀ in the low concentration groups are as shown in FIG. 5C, and the prediction results of C₄H₁₀ in the high concentration groups are as shown in FIG. 5D.

The dotted lines in FIG. 5A and FIG. 5D mark gas charging times. When the sample gases are just charged into a gas chamber, the mixed gas inside the gas chamber may be distributed nonuniformly to cause time response and transient fluctuation of the prediction result, which may last for about 30 minutes and then tend to be stable. Since the real-time spectra in the whole process are recorded at this continuous measurement and not subjected to subsequent average processing. Moreover, the experiment lasts for a long time and absorbance signals are inevitably affected by the drift of an absorption baseline. The prediction accuracy is reduced to different extents for both gases. For the low concentration groups, after stabilization, the maximum prediction error for C₃H₈ is 41 ppmv and the maximum prediction error for C₄H₁₀ is 45 ppmv. In the high concentration groups, after stabilization, the maximum prediction error for C₃H₈ is 26 ppmv and the maximum prediction error for C₄H₁₀ is 334 ppmv. Therefore, the prediction stability and relative reliability of the PLS model for the concentration of each component in the mixed gas of C₃H₈ and C₄H₁₀ are verified by continuous measurement experiments.

### 4. CONCLUSION

Due to the broadband absorption spectrum feature of mutual dense superposition of C₃H₈ and C₄H₁₀ over the near-infrared band, it is very difficult to accurately measure the concentrations of both gases on the spot in the oil gas industry. The present disclosure basically solves the technical problem of aliasing interference of broadband spectral lines by performing spectrum scanning in an area of 1685.9-1686.8 nm using a DFB laser, periodically demodulating second harmonic and direct absorption signals, and predicting the concentrations of C₃H₈ and C₄H₁₀ according to characteristic absorption thereof in the area. Real-time measurement of the concentrations of both single gases and the mixed gas is realized. Moreover, regardless of the single gas or the mixed gas, correlations of predicted values and true values of C₃H₈ and C₄H₁₀ are greater than 0.99. Even in the high concentration group of 2,000-10,000 ppmv, the prediction error may still be controlled below 148 ppmv, proving that the established concentration regression model has good prediction accuracy. In dynamic testing, the established prediction models also exhibit good dynamic reliability. The present disclosure provides a feasible and reliable solution for accurate measurement of the concentration of each component of oil gas volatiles, which has promising application expansion prospects and will be verified later in the field of oil and gas pipeline network leakage.

The foregoing are merely descriptions of preferred specific embodiments of the present disclosure, but the protection scope of the present disclosure is not limited thereto. Any equivalent replacement or modification made within a technical scope of the present disclosure by a person skilled in the art according to the technical solutions of the present disclosure and inventive concepts thereof shall fall within the protection scope of the disclosure.

What is claimed is:

1. A method for analyzing aliased spectra of alkane gases, comprising the following steps:
S1, separately acquiring second harmonic signals of specified gases at known gas concentrations and calculating an absorbance of each gas;
S2, constructing a low concentration prediction model regarding second harmonic signals and gas concentrations;
S3, constructing a high concentration prediction model regarding absorbances and gas concentrations;
S4, acquiring a second harmonic signal of a gas to be measured with an unknown gas concentration and calculating an absorbance of the gas to be measured; and
S5, if an absorbance peak of the gas to be measured is less than or equal to a threshold A₀, predicting the gas concentration of the gas to be measured using the low concentration prediction model; otherwise, predicting the gas concentration of the gas to be measured using the high concentration prediction model.

2. The method for analyzing aliased spectra of alkane gases according to claim 1, wherein the specified gases comprise a single gas C₃H₈, a single gas C₄H₁₀, and a mixed gas of the single gas C₃H₈ and the single gas C₄H₁₀ in a set ratio; and the threshold A₀ is 0.4.

3. The method for analyzing aliased spectra of alkane gases according to claim 2, wherein step S1 comprises the following specific steps:
S11, establishing an absorption cell having an optical path of L and emitting a triangular scanning signal from an incident end of the absorption cell into the absorption cell;
S12, subsequently bubbling N₂ into the absorption cell to create a N₂ atmosphere, and acquiring a N₂ second harmonic signal produced after irradiating N₂ with triangular scanning signals of different wavelengths, and taking the N₂ second harmonic signal as a light intensity baseline;
S13, subsequently bubbling a single gas and a mixed gas with known gas concentrations into the absorption cell separately;
S14, using triangular scanning signals of same wavelengths as the wavelengths in step S12 to individually irradiate the single gas and the mixed gas bubbled into the absorption cell, respectively, and obtaining a second harmonic signal formed after the irradiation; and
S15, finally calculating absorbances of each single gas and the mixed gas.

4. The method for analyzing aliased spectra of alkane gases according to claim 3, wherein the low concentration prediction model and the high concentration prediction model are each established by partial least squares regression.

5. The method for analyzing aliased spectra of alkane gases according to claim 4, wherein when the low concentration prediction model is established using partial least squares, a second harmonic signal is used as an independent variable and a low concentration independent variable matrix X₁^{n×m} is established, wherein n represents a number of samples; m represents a number of feature points, namely types of triangular scanning signal wavelengths; and with a gas concentration as a dependent variable, a dependent variable matrix Y₁^{n×p} is established, wherein p represents a category of a gas concentration.

6. The method for analyzing aliased spectra of alkane gases according to claim 5, wherein when the high concentration prediction model is established using partial least squares, an absorbance is used as an independent variable and a high concentration independent variable matrix X₂^{n×m} is established, wherein n represents a number of samples; m represents a number of feature points, namely types of triangular scanning signal wavelengths; and a gas concentration is used as a dependent variable and a dependent variable matrix Y₂^{n×p} is established, wherein p represents a category of a gas concentration.

7. The method for analyzing aliased spectra of alkane gases according to claim 6, wherein
a calculation formula for the absorbance of a gas with a known gas concentration is as follows:
$${A}_{1} - ln ( \frac{1}{exp [ - S ( T ) CPL φ ( v ) ]} ) ,$$
wherein A₁ represents the absorbance of the gas with the known gas concentration; v represents a wave number of an absorption line spectrum; S(T) represents a line intensity corresponding to a gas temperature of T; C represents the gas concentration; p represents a total pressure of the gas; φ(v) represents a linear absorption function of integral area normalization; and L represents the optical path of the absorption cell.

8. The method for analyzing aliased spectra of alkane gases according to claim 7, wherein
a calculation formula for the absorbance of a gas with an unknown gas concentration is as follows:
$${A}_{2} = ln ( \frac{{I}_{0}}{{I}_{t}} ) ,$$
wherein A₂ represents the absorbance of the gas with the unknown gas concentration; I₀ represents an initial light intensity of incident light; and Iₜ represents an outgoing light intensity of the incident light.

9. The method for analyzing aliased spectra of alkane gases according to claim 8, wherein a second harmonic signal is produced by the following process: a triangular scanning signal is emitted from the incident end of the absorption cell into the absorption cell to be absorbed by a gas, and then received and converted by a photoelectric detector integrated at an exit end of the absorption cell into an electric signal, and finally, the electric signal is demodulated by a lock-in amplifier to output the corresponding second harmonic signal.

10. The method for analyzing aliased spectra of alkane gases according to claim 9, wherein the absorbance peak of the gas to be measured is obtained by the following process: an infrared laser, used as a triangular scanning signal, is emitted from the incident end of the absorption cell into the absorption cell to be absorbed by a gas, and then received and converted by the photoelectric detector integrated at the exit end of the absorption cell into an infrared spectrum, and finally, the corresponding absorbance peak is obtained from the infrared spectrum.
